(19) United States
(12) Patent Application Publication
(10) Pub. No.: US 2023/0258670 A1
(43) Pub. Date: Aug. 17, 2023

(54) SENSORY TEST AND ENTRUSTED ANALYSIS METHOD

(71) Applicant: SHIMADZU CORPORATION, Kyoto (JP)

(72) Inventor: Keiko MATSUMOTO, Kyoto (JP)

(21) Appl. No.: 18/106,702

(22) Filed: Feb. 7, 2023

(30) Foreign Application Priority Data
Feb. 17, 2022 (JP) 2022-022784

(51) Int. Cl. G01N 35/00

(52) U.S. Cl. CPC G01N 35/00732; G01N 35/00603; G01N 2035/00821; G01N 2035/00891

(57) ABSTRACT

A sensory test and entrusted analysis method includes: receiving, by a second device, information identifying a sample, the information being transmitted from a first device; registering, by the second device, the information identifying the sample and a sensory evaluation item about the sample; adding, by the second device, identification information to the information identifying the sample; transmitting, by the second device, the identification information and an analysis item corresponding to the sensory evaluation item to a third device; receiving, by the second device, the identification information and an analysis result corresponding to the analysis item of the sample from the third device; registering, by the second device, a sensory result generated about the sample; and transmitting, by the second device, the analysis result and the sensory result to the first device.

### BACKGROUND OF THE INVENTION

### Field of the Invention

The present disclosure relates to a sensory test and entrusted analysis method.

### Description of the Background Art

As disclosed in Japanese Patent Laying-Open No. 2008-117365, there has been known a method for sending a specimen taken from a subject to a liaison company, transferring the sent specimen to a management company, and sending the specimen from the management company to each examination company based on an examination type of the specimen.

### SUMMARY OF THE INVENTION

The method disclosed in Japanese Patent Laying-Open No. 2008-117365 is a method for exchanging the specimen and various types of information about the specimen among the subject, the liaison company, the management company, and the examination companies, and the procedure is complicated. In addition, the method disclosed in Japanese Patent Laying-Open No. 2008-117365 is not a method in the field that deals with a food sample. In the field that deals with a food sample, when a customer makes a request for analysis of a food sample, the customer sends analysis items and the food sample to an entrusted analysis company, and the entrusted analysis company returns an analysis result, and the customer makes a payment. When a. customer makes a request for a sensory test of the food sample, the customer sends sensory test items and the food sample to a sensory test company, the sensory test company returns a sensory result to the customer, and the customer makes a payment.

When support for the sensory test is required, a request is separately made of an organization where an expert is present to obtain a consideration result based on the analysis result and the sensory result, and the customer makes a payment. As described above, in the field that deals with a food sample, the procedure is complicated, and thus, there has been a demand for a method for efficiently exchanging information about an analysis result of a food sample and a sensory result of the food sample.

The present disclosure has been made to solve the above-described problem, and an object thereof is to provide a sensory test and entrusted analysis method that allows efficient exchange of information among a customer, a system management company and an entrusted analysis company.

The present disclosure relates to a sensory test and entrusted analysis method. performed among a first device, a second device and a third device, the first device being usable by a customer, the second device being usable by a system management company, the third device being usable by an entrusted analysis company including an analytical instrument for analyzing a sample. The sensory test and entrusted analysis method includes: receiving, by the second device, information identifying the sample, the information being transmitted from the first device; registering, by the second device, the information identifying the sample and a sensory evaluation item about the sample; adding, by the second device, identification information to the information identifying the sample; transmitting, by the second device, the identification information and an analysis item corresponding to the sensory evaluation item to the third device; receiving, by the second device, the identification information and an analysis result corresponding to the analysis item of the sample from the third device; registering, by the second device, a sensory result generated about the sample; and transmitting, by the second device, the analysis result and the sensory result to the first device.

According to the present disclosure, there can be provided a sensory test and entrusted analysis method that allows efficient exchange of information among a customer, a system management company and an entrusted analysis company.

The foregoing and other objects, features, aspects and advantages of the present disclosure will become more apparent from the following detailed description of the present disclosure when taken in conjunction with the accompanying drawings.

### BRIEF DESCRIPTION OF THE DRAWINGS

FIG. 1 shows an overall configuration of an analysis system according to a first embodiment.

FIG. 2 is a diagram for illustrating a flow of the analysis system according to the first embodiment.

FIG. 3 shows a configuration of each device of the analysis system according to the first embodiment.

FIG. 4 is a sequence diagram of the analysis system according to the first embodiment.

FIG. 5 shows a relationship between standard sensory evaluation items and analysis items.

FIG. 6 shows information associated with IDs.

FIG. 7 shows sensory results and analysis results about food samples.

FIG. 8 shows a configuration of each device of an analysis system according to a second embodiment.

FIG. 9 is a diagram for illustrating learning of an estimation model in a learning phase.

FIG. 10 shows a configuration of the analysis system in an operation phase.

FIG. 11 is a diagram for illustrating a flow of an analysis system according to a third embodiment.

FIG. 12 is a sequence diagram of the analysis system according to the third embodiment.

### DESCRIPTION OF THE PREFERRED EMBODIMENTS

Embodiments will be described in detail with reference to the drawings, in which the same or corresponding portions are denoted by the same reference characters and description thereof will not be repeated in principle.

### First Embodiment

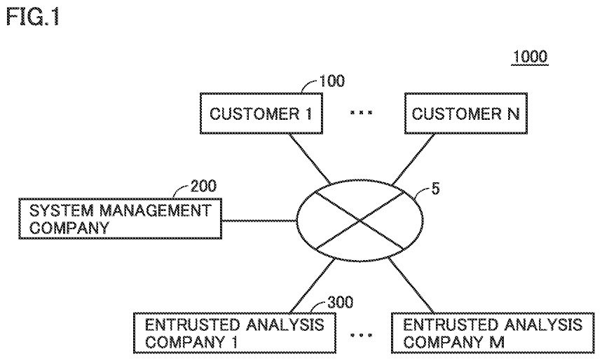

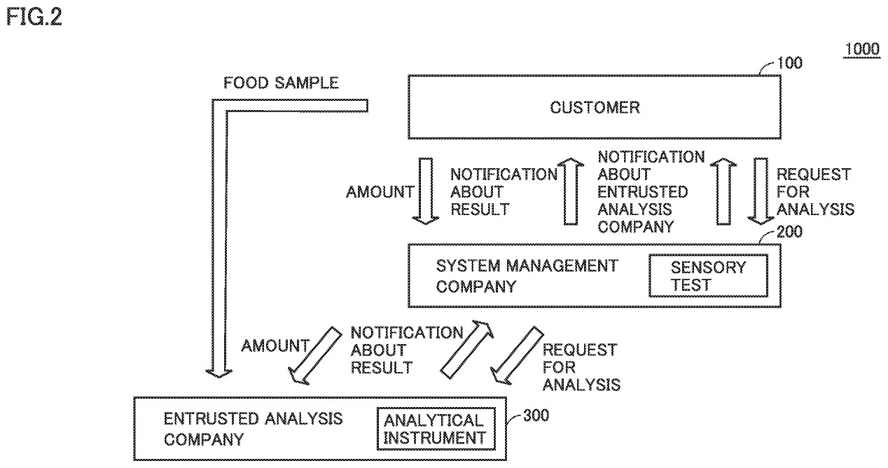

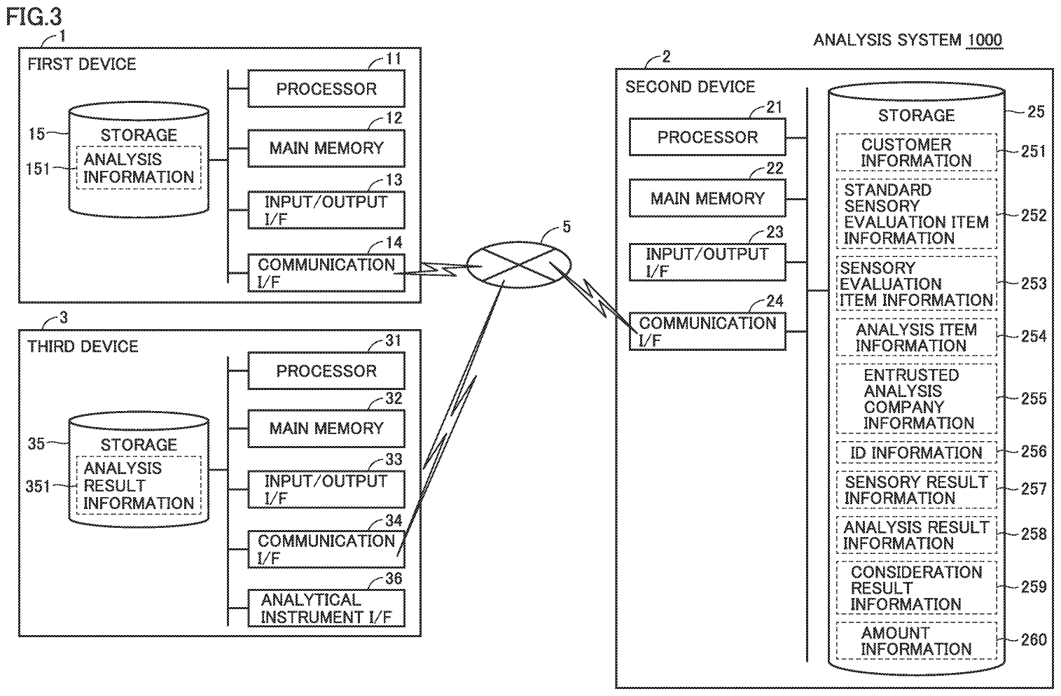

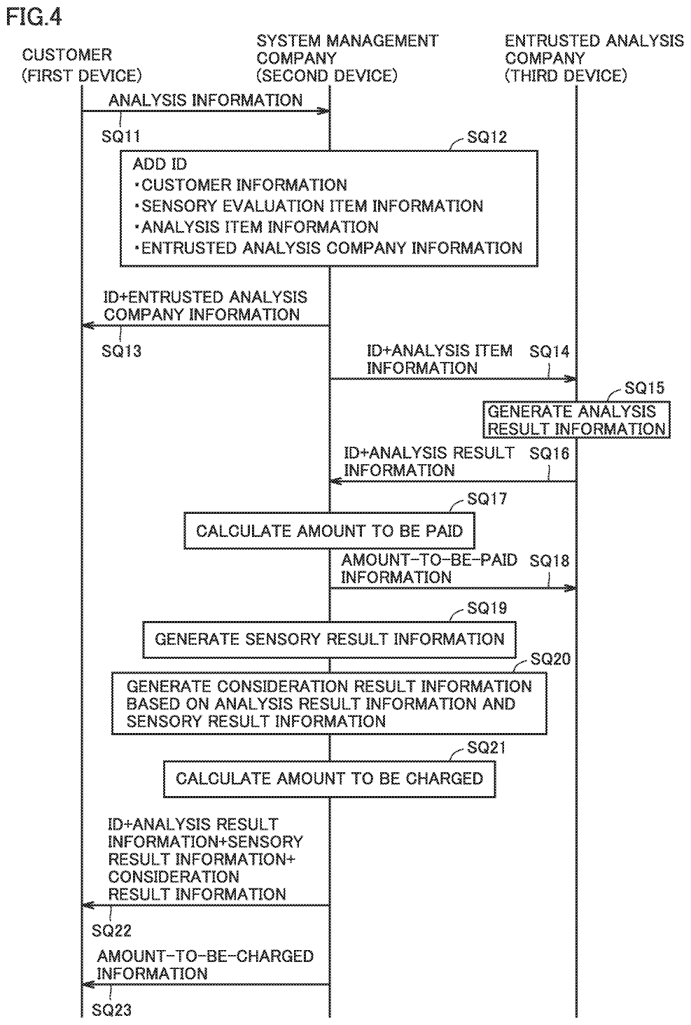

A sensory test and entrusted analysis method in an analysis system 1000 according to a first embodiment will be described with reference to FIGS. 1 to 4. FIG. 1 shows an overall configuration of analysis system 1000 according to the first embodiment. FIG. 2 is a diagram for illustrating a flow of analysis system 1000 according to the first embodiment. FIG. 3 shows a configuration of each device of analysis system 1000 according to the first embodiment. FIG. 4 is a sequence diagram of analysis system 1000 according to the first embodiment.

As shown in FIG. 1, in analysis system 1000, a device of a customer 100, a device of a system management company 200, and a device of an entrusted analysis company 300 are connected through a network 5. Customer 100 makes a request for analysis of a food sample, and includes a customer 1 to a customer N, for example. System management company 200 manages the whole of analysis system 1000, and includes one system management company, for example. Entrusted analysis company 300 analyzes the food sample, and includes an entrusted analysis company 1 to an entrusted analysis company M, for example. Entrusted analysis company 300 may include one entrusted analysis company.

The flow of analysis system 1000 among customer 100, system management company 200 and entrusted analysis company 300 will be described with reference to FIG. 2. Customer 100 makes a request of system management company 200 to analyze a food sample. Based on the request for analysis, system management company 200 determines entrusted analysis company 300 including an analytical instrument, and notifies customer 100 about determined entrusted analysis company 300. Customer 100 sends the food sample to determined entrusted analysis company 300.

System management company 200 makes a request of entrusted analysis company 300 to analyze the food sample. Entrusted analysis company 300 analyzes the food sample using the analytical instrument. Entrusted analysis company 300 notifies system management company 200 about an analysis result. System management company 200 causes a sensory evaluator belonging to system management company 200 to conduct a sensory test of the food sample. The sensory test refers to a test for determining the quality of a product using the human senses (such as a sense of sight, a sense of hearing, a sense of taste, a sense of smell, and a sense of touch), and is conducted on foodstuffs, flavorings, industrial products and the like.

System management company 200 notifies customer 100 about the analysis result, a sensory result, and a consideration result generated based on the analysis result and the sensory result. System management company 200 makes a payment for the request for analysis to entrusted analysis company 300, Customer 100 makes a payment for the request for analysis to system management company 200.

A configuration of each device used in analysis system 1000 will be described with reference to FIG. 3. in analysis system 1000, a first device 1 of customer 100, a second device 2 of system management company 200, and a third device 3 of entrusted analysis company 300 are connected through network 5.

Each of first device 1, second device 2 and third device 3 is configured in accordance with a general-purpose computer architecture. In the present embodiment, each of first device 1, second device 2 and third device 3 is implemented by a desktop-type computer. Each of first device 1, second device 2 and third device 3 may be implemented by a device other than the desktop-type computer, such as a laptop-type computer, a tablet-type computer, or a mobile terminal including a smartphone.

First device 1 includes a processor 11, a main memory 12, an input/output interface 13, a communication interface 14, and a storage 15. These components are connected thorough a bus.

Processor 11 is a main component for computation (computer) that performs various types of processing in accordance with various types of programs. Processor 11 is implemented by, for example, at least one of a central processing unit (CPU), a field programmable gate array (FPGA), a graphics processing unit (GPU), and a multi processing unit (MPU). Processor 11 may be implemented by a processing circuitry. Processor 11 reads a program stored in storage 15 and loads the program into main memory 12 for execution.

Main memory 12 is implemented by, for example, a volatile storage device such as a random access memory (RAM), a dynamic RAM (DRAM) or a static random access memory (SRAM), or a nonvolatile storage device such as a read only memory (ROM).

Input/output interface 13 communicates, to each component, an input signal by a user of a button, a touch panel or the like, and an output signal to a display device such as a liquid crystal display.

Communication interface 14 receives and transmits data (information) to and from another device, using wired connection or wireless connection. In the present embodiment, communication interface 14 receives and transmits data (information) to and from a communication interface 24 of second device 2, using wireless communication through network 5.

Storage 15 is implemented by, for example, a nonvolatile storage device such as a hard disk drive (HDD) or a solid state drive (SSD). Storage 15 stores analysis information 151 and the like as information specifying a sample, the information including sensory evaluation items for the sensory test of the food sample, and the like.

Second device 2 includes a processor 21, a main memory 22, an input/output interface 23, a communication interface 24, and a storage 25. These components are connected through a bus.

Configurations of processor 21, main memory 22, input/output interface 23, communication interface 24, and storage 25 in second device 2 are the same as those of processor 11, main memory 12, input/output interface 13, communication interface 14, and storage 15 in first device 1.

Storage 25 stores customer information 251, standard sensory evaluation item information 252, sensory evaluation item information 253, analysis item information 254, entrusted analysis company information 255, ID information 256, sensory result information 257, analysis result information 258, consideration result information 259, amount information 260 and the like.

Customer information 251 is information about customer 100. Standard sensory evaluation item information 252 is information about standard sensory evaluation items that are predetermined for each specific food sample. Sensory evaluation item information 253 includes sense-of-taste information such as, for example, sweetness and sourness, sense-of-smell information and the like. Analysis item information 254 is information about analysis items for the food sample transmitted from first device 1. Entrusted analysis company information 255 is information about entrusted analysis company 300 determined based on the analysis items.

ID information 256 is information for identifying the customer, which is added in association with customer information 251. Sensory result information 257 is information about the result of the sensory test. Analysis result information 258 is information about the analysis result of the food sample. Consideration result information 259 is information indicating a relationship between sensory result information 257 and analysis result information 258. Amount information 260 is information about an amount to be paid to entrusted analysis company 300 and an amount to be charged to customer 100.

Third device 3 includes a processor 31, a main memory 32, an input/output interface 33, a communication interface 34, a storage 35, and an analytical instrument interface 36. These components are connected through a bus.

Configurations of processor 31, main memory 32, input/output interface 33, communication interface 34, and storage 35 in third device 3 are the same as those of processor 11, main memory 12, input/output interface 13, communication interface 14, and storage 15 in first device 1.

Analytical instrument interface 36 inputs and outputs information to and from the analytical instrument of entrusted analysis company 300 for analyzing the food sample. Storage 35 stores analysis result information 351 about the analysis result of the food sample, and the like.

A sensory test and entrusted analysis method performed among first device 1 of customer 100, second device 2 of system management company 200, and third device 3 of entrusted analysis company 300 will be described with reference to FIG. 4. “SQ” in FIG. 4 refers to a sequence.

In SQ11, first device 1 transmits the analysis information to second device 2. In SQ12, second device 2 that has received the analysis information associates, with one piece of ID information, the customer information and the entrusted analysis company information, together with the sensory evaluation item information included in the analysis information and the analysis item information obtained from the sensory evaluation item information. The sensory evaluation item information about the sample may be obtained by system management company 200 and input to and registered with second device 2. The analysis item information may be transmitted from first device 1 to second device 2.

In SQ13, second device 2 transmits the ID information and the entrusted analysis company information to first device 1. When a commercially available product or the like is analyzed without receiving any sample from customer 100, the information does not need to be transmitted from second device 2 to first device 1. In SQ14, second device 2 transmits the ID information and the analysis item information to third device 3. In SQ15, third device 3 analyzes the food sample using the analytical instrument, based on the received analysis item information. Thereafter, in SQ15 third device 3 generates the analysis result information. In SQ16, third device transmits the ID information and the analysis result information to second device 2. The customer may send the sample to the entrusted analysis company, based on the transmitted entrusted analysis company information.

In SQ17, based on the analysis result information, second device 2 calculates an amount to be paid, which is an amount to be paid to entrusted analysis company 300. In SQ18, second device 2 transmits the amount-to-be-paid information calculated in SQ17 to third device 3. A payment itself may be separately made by money transfer 25 from system management company 200 to an account or the like of entrusted analysis company 300. The amount to be paid may be calculated by third device 3 of entrusted analysis company 300 and the amount-to-be-paid information may be transmitted from third device 3 to second device 2.

In SQ19, second device 2 generates the sensory result information, based on the sensory test result separately obtained from the sensory evaluator. In SQ20, second device 2 generates the consideration result information based on the analysis result information and the sensory result information. The consideration result information is, for example, data indicating a relationship between a peak value in a graph of the analysis result and a taste corresponding to the peak value.

In SQ21, second device 2 calculates an amount to be charged, which is an amount to be charged to customer 100. In SQ22, second device 2 transmits the ID information, the analysis result information, the sensory result information, and the consideration result information to first device 1. When a commercially available product or the like is analyzed without receiving any sample from customer 100, the ID information does not need to be transmitted from second device 2 to first device 1. In SQ23, second device 2 transmits the amount-to-be-charged information calculated in SQ21 to first device 1. A payment itself may be separately made by money transfer from customer 100 to an account or the like of system management company 200.

According to the sensory test and entrusted analysis method shown in FIG. 4, second device 2 of system management company 200 can manage the information received and transmitted to and from first device 1 of customer 100 and third device 3 of entrusted analysis company 300. Thus, as compared with the case in which customer 100 directly exchanges the information with entrusted analysis company 300, more efficient exchange of the information can be achieved because system management company 200 manages the information in a centralized manner.

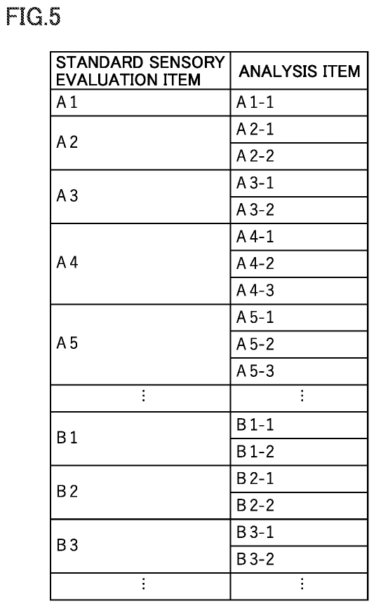

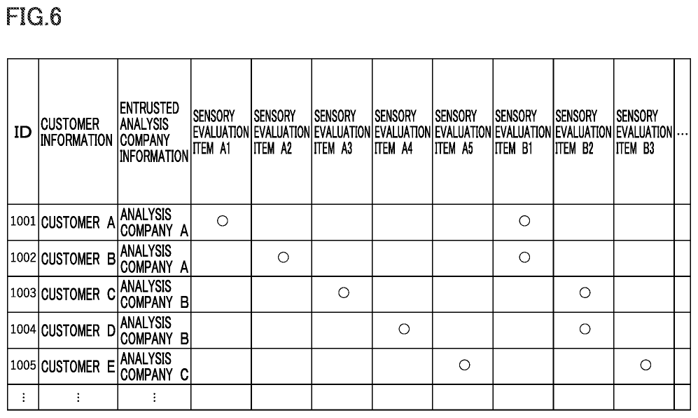

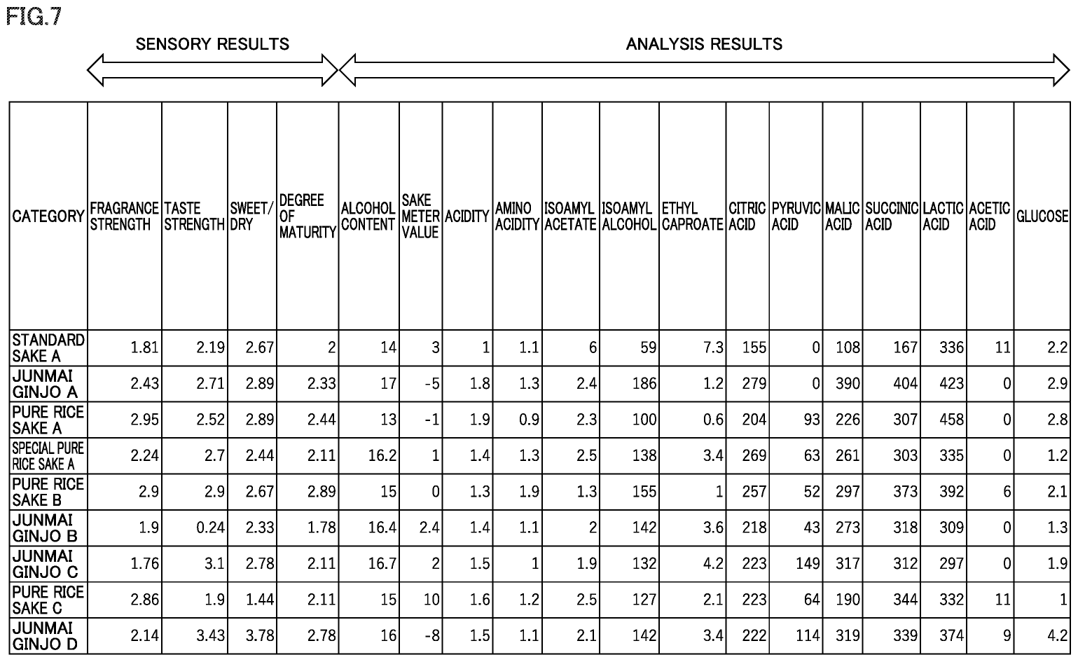

The information stored in storages 15, 25 and 35 will be specifically described with reference to FIGS. 5 to 7. FIG. 5 shows a relationship between standard sensory, evaluation items and analysis items. FIG. 6 shows information associated with IDs. FIG. 7 shows sensory results and analysis results about food samples.

As shown in FIG. 5, each of the standard. sensory evaluation items is associated with one or more analysis items. For example, a standard sensory evaluation item “A1” is associated with an analysis item “A1-1” a standard sensory evaluation item “A2” is associated with analysis items “A2-1” and “A2-2”, a standard sensory evaluation item “A3” is associated with analysis items “A3-1” and “A3-2”, a standard sensory evaluation item “A4” is associated with analysis items “A.4-1”, “A4-2” and “A4-3”, and a standard sensory evaluation item “A5” is associated with analysis items “A5-1”, “A5-2” and “A5-3”. As described above, second device 2 includes a database that stores the standard sensory evaluation items shown in FIG. 5 and the one or more analysis items corresponding to the standard sensory evaluation items in association with each other.

As shown in FIG. 6, second device 2 stores IDs for identifying customers and various items in association with each other. A circle mark in a sensory evaluation item section indicates a sensory evaluation item that each customer wants to check. Thus, a sensory evaluation item that each customer wants to check is selected from the standard sensory evaluation items. An item indicated by a circle mark in a sensory evaluation item section is associated with one or more analysis items as shown in FIG. 5. That is, by selecting sensory evaluation items from the standard sensory evaluation items, analysis items are set.

As shown in FIG. 6, an ID “1001” is associated with customer information “customer A”, entrusted analysis company information “analysis company A”, and sensory evaluation items “A1” and “B1”. An ID “1002” is associated with customer information “customer.”, entrusted analysis company information “analysis company A”, and sensory evaluation items “A2” and “B1”. An ID “1003” is associated with customer information “customer C”, entrusted analysis company information “analysis company B”, and sensory evaluation items “A3” and “B2”. An ID “1004” is associated with customer information “customer D”, entrusted analysis company, information “analysis company B”, and sensory evaluation items “A4” and “B2”. An ID “1005” is associated with customer information “customer E”, entrusted analysis company information “analysis company C”, and sensory evaluation items “A5” and “B3”.

Second device 2 selects, from a plurality of entrusted analysis companies, an entrusted analysis company including an analytical instrument that can analyze analysis items corresponding to sensory evaluation items that a customer wants to check. Second device 2 may also take a degree of busyness of each entrusted analysis company and the like into consideration, when selecting the entrusted analysis company. When there are many analysis items, or when the use of a plurality of analytical instruments is required, second device 2 may select a plurality of entrusted analysis companies.

FIG. 7 shows a specific example of sensory results and analysis results when a plurality of types of Japanese sake are used as food samples. In the case of Japanese sake, for example, a sensory evaluator conducts a sensory test to thereby quantify the sensory evaluation items “fragrance strength”, “taste strength”, “sweet/dry”, and “degree of maturity”. As analysis items corresponding to these sensory evaluation items. “alcohol content”, “sake meter value”, “acidity”, “amino acidity”, “isoamyl acetate”, “isoamyl alcohol”, “ethyl caproate”, “citric acid”, “pyruvic acid”, “malic acid”, “succinic acid”, “lactic acid”, “acetic acid”, and “glucose” are analyzed and quantified by an analytical instrument.

More specifically, for example, the sensory evaluation item “fragrance strength” is affected by the amounts of the analysis items “ethyl caproate” and “glucose”. When these amounts are large, “fragrance strength” can be determined as strong.

Based on the analysis results and the sensory results shown in FIG. 7, second device 2 generates a consideration result indicating a relationship between the analysis results and the sensory results. The consideration result is, for example, data. indicating that a peak value of a chromatogram of an analysis result corresponds to an item of a taste in sensory evaluation.

### Second Embodiment

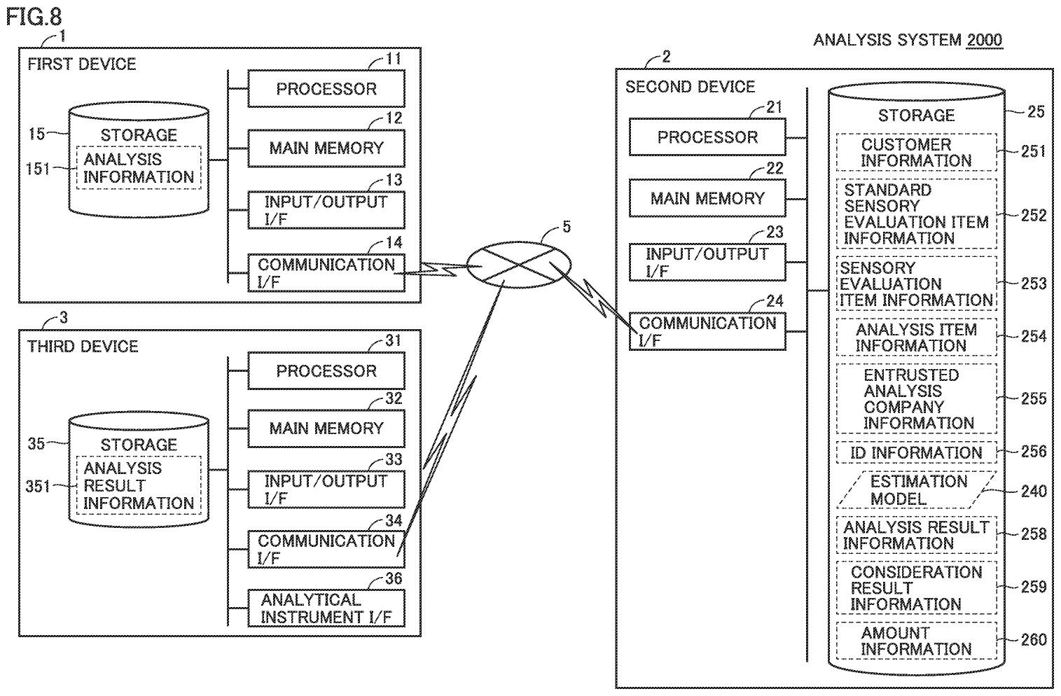

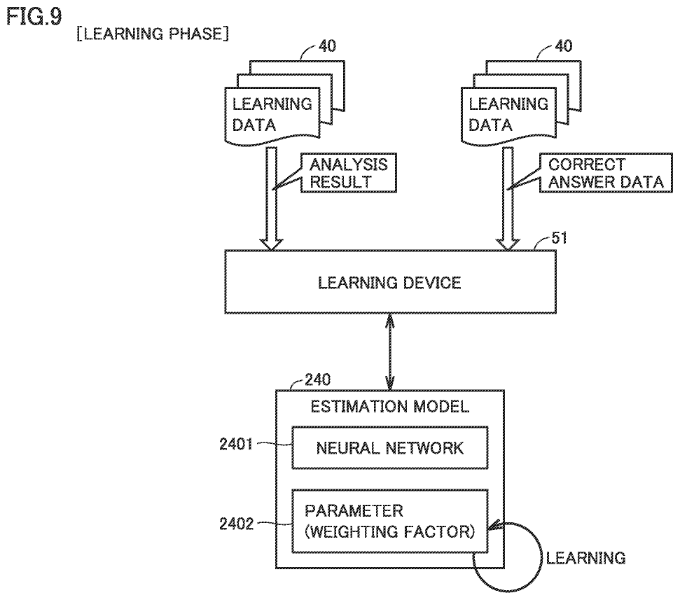

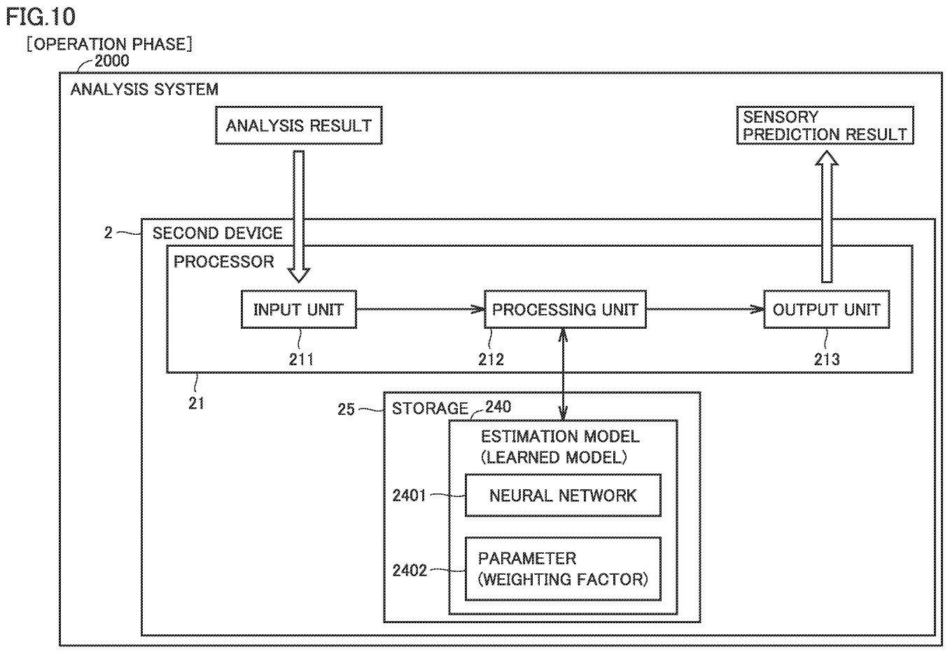

A sensory test and entrusted analysis method in an analysis system 2000 according to a second embodiment will be described with reference to FIGS. 8 to 10. FIG. 8 shows a configuration of each device of analysis system 2000 according to the second embodiment. FIG. 9 is a diagram for illustrating learning of an estimation model in a learning phase. FIG. 10 shows a configuration of analysis system 2000 in an operation phase.

Analysis system 2000 according to the second embodiment is different from analysis system 1000 according to the first embodiment in that sensory result information 257 is replaced with an estimation model 240, and the remaining basic configuration of analysis system 2000 is the same as that of analysis system 1000. The learning phase and the operation phase of estimation model 240 will be described below.

As shown in FIG. 9, the learning phase is a prior learning phase in which estimation model 240 is learned before an analysis result is provided to second device 2. Estimation model 240 is learned by a learning device 51 to estimate a sensory result from the analysis result.

A known algorism such as supervised learning, unsupervised learning and reinforcement learning can be used as a learning algorithm for learning estimation model 240. In the present embodiment, learning device 51 causes estimation model 240 to be learned through supervised learning using learning data 40.

Learning data 40 is preliminarily prepared for learning of estimation model 240 and includes an analysis result and a sensory result corresponding to the analysis result. For example, a program designer associates a plurality of analysis results in the past about a food sample with sensory results (correct answer data) corresponding to the analysis results, to thereby form learning data 40. The designer preliminarily prepares a plurality of pieces of such learning data 40.

Estimation model 240 includes a neural network 2401 and a parameter 2402 used by neural network 2401. A known neural network used in the processing by deep learning such as a convolution neural network (CNN), a recurrent neural network (RNN) or long short term memory (LSTM) network is applied as neural network 2401.

Estimation model 240 uses above-described neural network 2401 to perform deep learning. Parameter 2402. includes a weighting factor or the like used for calculation by neural network 2401. Estimation model 240 is not limited to a model learned through deep learning using the neural network, and may be a model learned through another type of machine learning.

Learning device 51 receives an input of the analysis result, of learning data 40. Learning device 51 performs a process for estimating the sensory result based on the input analysis result and estimation model 240 including neural network 2401.

The operation phase shown in FIG. 10 is a phase in which the sensory result is estimated using estimation model 240 after the analysis result is provided. As shown in FIG. 10, second device 2 stores, in storage 25, estimation model 240 learned by learning device 51 shown in FIG. 9. For example, second device 2 obtains estimation model 240 from learning device 51, and stores obtained estimation model 240 in storage 25. Learning device 51 may be second device 2 and the above-described function of learning device 51 may be a function of processor 21 of second device 2.

Processor 21 of second device 2 includes an input unit 211, a processing unit 212 and an output unit 213. Input unit 211 receives the input of the analysis result. Processing unit 212 performs the process for estimating the sensory result based on the analysis result input from input unit 211 and estimation model 240 including neural network 2401. As described above, estimation model 240 is not limited to a model learned. through deep learning using the neural network, and may be a model learned through another type of machine learning. Output unit 213 outputs the sensory result obtained by processing unit 212 as a sensory prediction result.

### Third Embodiment

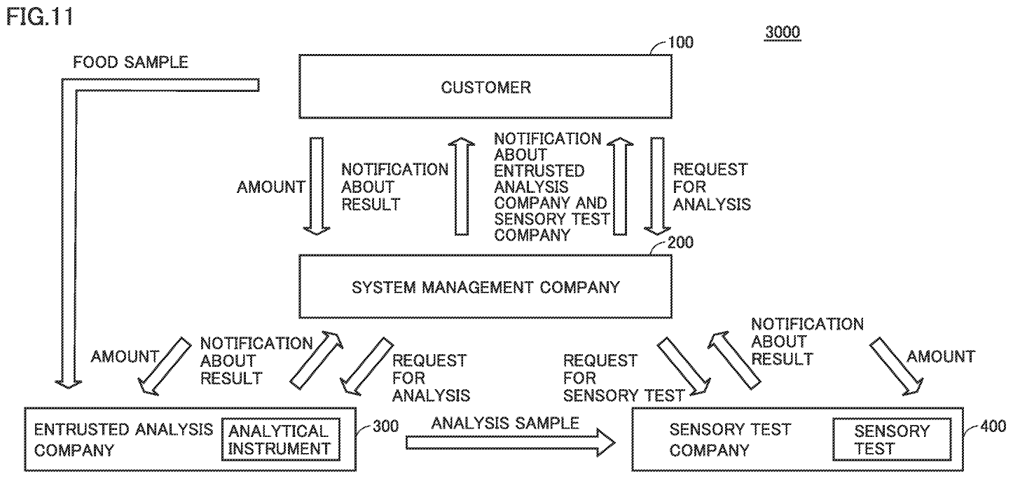

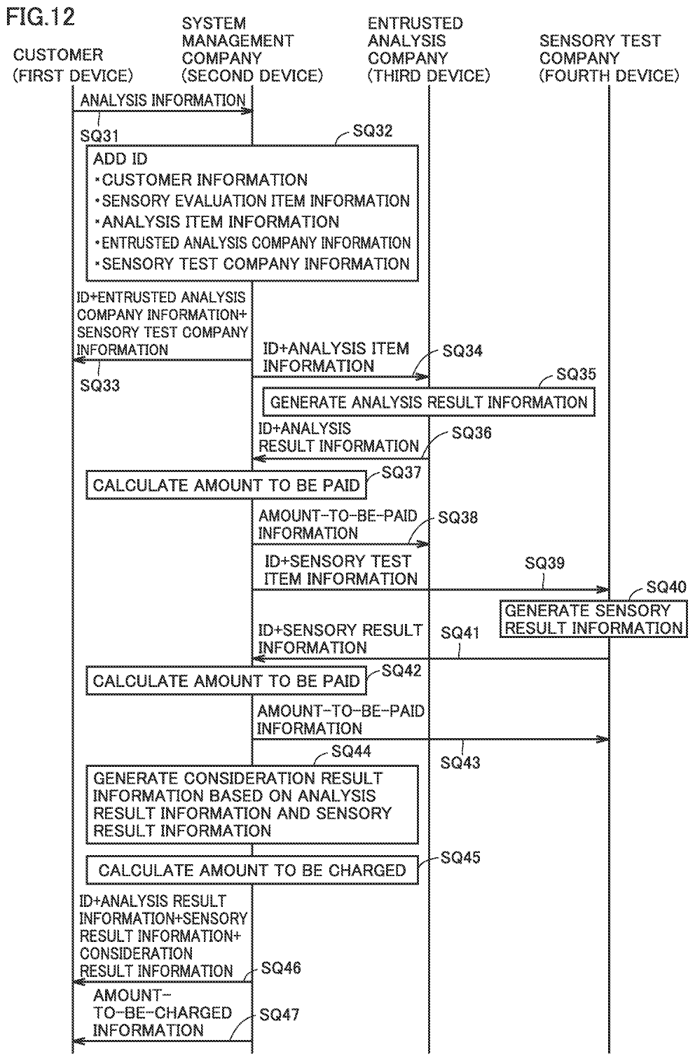

A sensory test and entrusted analysis method in an analysis system 3000 according to a third embodiment will be described with reference to FIGS. 11 to 12. FIG. 11 is a diagram for illustrating a flow of analysis system 3000 according to the third embodiment. FIG. 12 is a sequence diagram of analysis system 300 according to the third embodiment.

As shown in FIG. 11 in analysis system 3000, a sensory test company 400 that conducts a sensory test is provided separately. The flow of analysis system 3000 among customer 100, system management company 200, entrusted analysis company 300, and sensory test company 400 will be described with reference to FIG. 11. Customer 100 makes a request of system management company 200 to analyze a food sample. Based on the request for analysis, system management company 200 determines entrusted analysis company 300 including an analytical instrument, and determines sensory test company 400 of which system management company 200 can make a request to conduct a sensory test.

System management company 200 notifies customer 100 about determined entrusted analysis company 300 and sensory test company 400. Customer 100 sends the food sample to determined entrusted analysis company 300. System management company 200 makes a request of entrusted analysis company 300 to analyze the food sample. Entrusted analysis company 300 analyzes the food sample using the analytical instrument. Entrusted analysis company 300 notifies system management company 200 about an analysis result. System management company 200 makes a payment for the request for analysis to entrusted analysis company 300.

System management company 200 makes a request of entrusted analysis company 300 to analyze the food sample. Entrusted analysis company 300 sends the food sample to sensory test company 400. Customer 100 may preliminarily send the food sample to sensory test company 400. Sensory test company 400 causes a sensory evaluator belonging to sensory test company 400 to conduct a sensory test of the food sample. Sensory test company 400 notifies system management company 200 about a sensory result. System management company 200 makes a payment for the request for the sensory test to sensory test company 400.

System management company 200 notifies customer 100 about the analysis result, the sensory result, and a consideration result generated based on the analysis result and the sensory result. Customer 100 makes a payment for the request for analysis to system management company 200.

A sensory test and entrusted analysis method among first device 1 of customer 100, second device 2 of system management company 200, third device 3 of entrusted analysis company 300, and a fourth device 4 of the sensory test company will be described with reference to FIG. 12.

In SQ31, first device 1 transmits the analysis information to second device 2. In SQ32, second device 2 that has received the analysis information associates, with one piece of ID information, the customer information, the entrusted analysis company information and the sensory test company information, together with the sensory evaluation item information included in the analysis information and the analysis item information obtained from the sensory evaluation item information.

In SQ33, second device 2 transmits the ID information, the entrusted analysis company information and the sensory test company information to first device 1. In SQ34, second device 2 transmits the ID information and the analysis item information to third device 3, In SQ35, third device 3 analyzes the food sample using the analytical instrument, based on the received analysis item information. Thereafter, in SQ35, third device 3 generates the analysis result information. In SQ36, third device 3 transmits the ID information and the analysis result information to second device 2.

In SQ37, second device 2 calculates a first amount to be paid, which is an amount to be paid to entrusted analysis company 300, based on the analysis result information. In SQ38, second device 2 transmits the first-amount-to-be-paid information calculated in SQ37 to third device 3. A payment itself may be separately made by money transfer from system management company 200 to an account or the like of entrusted analysis company 300. The amount to be paid may be calculated by third device 3 of entrusted analysis company 300 and the amount-to-be-paid information may be transmitted from third device 3 to second device 2.

In SQ39, second device 2 transmits the ID information and the sensory test item information to fourth device 4. In SQ40, fourth device 4 generates the sensory result information based on the input sensory test result obtained from the sensory evaluator. In SQ41, fourth device 4 transmits the ID information and the sensory result information to second device 2.

In SQ42, second device 2 calculates a second amount to be paid, which is an amount to be paid to sensory test company 400, based on the sensory result information. In SQ43, second device 2 transmits the second-amount-to-be-paid information calculated in SQ42 to fourth device 4. A payment itself may be separately made by money transfer from system management company 200 to an account or the like of sensory test company 400. The amount to be paid may be calculated by fourth device 4 of sensory test company 400 and the amount-to-be-paid information may be transmitted from fourth device 4 to second device 2.

In SQ44, second device 2 generates the consideration result information based on the analysis result information and the sensory result information. The consideration result information is, for example, data indicating a relationship between a peak value in a graph of the analysis result and a taste corresponding to the peak value.

In SQ45, second device 2 calculates an amount to be charged to customer 100. In SQ46, second device 2 transmits the information, the analysis result information, the sensory result information, and the consideration result information to first device 1. In SQ47, second device 2 transmits the amount-to-be-charged information calculated in SQ45 to first device 1. A payment itself may be separately made by money transfer from customer 100 to an account or the like of system management company 200.

<Modifications>

In each of the above-described embodiments, description has been given of the case in which the sensory test is conducted in system management company 200 or sensory test company 400. The sensory test may be conducted in customer 100 or entrusted analysis company 300 including the analytical instrument. For example, when the sensory test is conducted in customer 100, the amount to be charged to customer 100 may be reduced accordingly.

In each of the above-described embodiments, third device 3 of entrusted analysis company 300 may transmit the analysis result directly to first device 1 of customer 100, without transmitting the analysis result to second device 2 of system management company 200. That is, the reception and transmission of the analysis result by second device 2 of system management company 200 can be omitted.

In each of the above-described embodiments, first device 1 of customer 100 may include a database that stores the standard sensory evaluation items and the one or more analysis items corresponding to the standard sensory evaluation items in association with each other. First device 1 may set the analysis items based on the sensory evaluation items by using the database, and transmit the set information to second device 2.

In each of the above-described embodiments, the analysis information transmitted from first device 1 to second device 2 may include information about the manufacturing date of the food sample, a type of the food sample, and the like.

### ASPECTS

It is understood by those skilled in the art that the plurality of exemplary embodiments described above are specific examples of the following aspects.

(Clause 1) A sensory test and entrusted analysis method according to one aspect relates to a sensory test and entrusted analysis method performed among a first device, a second device and a third device, the first device being usable by a customer, the second device being usable by a system management company, the third device being usable by an entrusted analysis company including an analytical instrument for analyzing a sample. The sensory test and entrusted analysis method includes: receiving, by the second device, information identifying the sample, the information being transmitted from the first device; registering, by the second device, the information identifying the sample and a sensory evaluation item about the sample; adding; by the second device, identification information to the information identifying the sample; transmitting, by the second device, the identification information and an analysis item corresponding to the sensory evaluation item to the third device; receiving, by the second device, the identification information and an analysis result corresponding to the analysis item of the sample from the third device; registering, by the second device, a sensory result generated about the sample; and transmitting, by the second device, the analysis result and the sensory result to the first device.

According to the sensory test and entrusted analysis method of clause 1, the information can be efficiently exchanged among customer, the system management company and the entrusted analysis company.

(Clause 2) The sensory evaluation item is selected from a standard sensory evaluation item. The standard sensory evaluation item is associated with the one or more analysis items. The second device includes a database that stores the standard sensory evaluation item and the one or more analysis items corresponding to the standard sensory evaluation item in association with each other. The sensory test and entrusted analysis method further includes setting, by the second device, the analysis item corresponding to the sensory evaluation item by using the database.

According to the sensory test and entrusted analysis method of clause 2, by, selecting the sensory evaluation item obtained from the standard sensory evaluation item, the desired analysis item corresponding to the sensory evaluation item can be easily set.

(Clause 3) A fourth device usable by a sensory test company that conducts a sensory test is further provided. The sensory test and entrusted analysis method further includes: transmitting, by the second device, the identification information and a sensory test item corresponding to the sensory evaluation item to the fourth device; and receiving, by the second device, the sensory result transmitted from the fourth device.

According to the sensory test and entrusted analysis method of clause 3, the information can be efficiently exchanged even when the sensory test company conducts the sensory test.

(Clause 4) The second device generates the sensory result corresponding to the sensory evaluation item.

According to the sensory test and entrusted analysis method of clause 4, the system management company generates the sensory result, and thus, the sensory result can be efficiently transmitted to the customer.

(Clause 5) The second device includes a machine learning device and a storage device that stores the analysis result in the past as learning data. The sensory test and entrusted analysis method further includes: generating, by the machine learning device, the sensory result by using the learning data; and transmitting, by the second device, the generated sensory result to the first device.

According to the sensory test and entrusted analysis method of clause 5, the machine learning device is used; and thus; the more accurate sensory result can be obtained as compared with the case of not using the machine learning device.

(Clause 6) The sensory test and entrusted analysis method further includes: calculating, by the second device, an amount to be paid, the amount to be paid being an amount to be paid to the entrusted analysis company for the analysis result; and calculating, by the second device, an amount to be charged based on the amount to be paid the amount to be charged being an amount to be charged to the customer.

According to the sensory test and entrusted analysis method of clause 6; the second device of the system management company calculates the amounts, and thus, management of the amounts can become easy.

(Clause 7) The sensory test and entrusted analysis method further includes: receiving, by the second device, an amount to be paid from the third device, the amount to be paid being an amount to be paid to the entrusted analysis company for the analysis result; and calculating, by the second device, an amount to be charged based on the amount to be paid; the amount to be charged being an amount to be charged to the customer.

According to the sensory test and entrusted analysis method of clause 7; the second device of the system management company calculates the amount to be charged, and thus, management of the amount can become easy.

(Clause 8) The sensory test and entrusted analysis method further includes: calculating, by the second device, a first amount to be paid, the first amount to be paid being an amount to be paid to the entrusted analysis company for the analysis result; calculating, by the second device, a second amount to be paid, the second amount to be paid being an amount to be paid to the sensory test company for the sensory result; and calculating, by the second device, an amount to be charged based on the first amount to be paid and the second amount to be paid, the amount to be charged being an amount to be charged to the customer.

According to the sensory test and entrusted analysis method of clause 8, the second device of the system management company calculates the amounts, and thus, management of the amounts can become easy.

(Clause 9) The sensory test and entrusted analysis method further includes: receiving, by the second device, a first amount to be paid from the third device, the first amount to be paid being an amount to be paid to the entrusted analysis company for the analysis result; receiving, by the second device, a second amount to be paid from the fourth device, the second amount to be paid being an amount to be paid to the sensory test company for the sensory result; and calculating, by the second device, an amount to be charged based on the first amount to be paid and the second amount to be paid, the amount to be charged being an amount to be charged to the customer.

According to the sensory test and entrusted analysis method of clause 9, the second device of the system management company calculates the amount to be charged, and thus, management of the amount can become easy.

(Clause 10) The sensory test and entrusted analysis method further includes: generating, by the second device, a consideration result based on the analysis result and the sensory result, the consideration result indicating a relationship between the analysis result and the sensory result; and transmitting, by the second device, the consideration result to the first device.

According to the sensory test and entrusted analysis method of clause 10, the second device of the system management company generates the consideration result and transmits the generated consideration result to the first device, and thus, the information can be efficiently exchanged.

(Clause 11) A sensory test and entrusted analysis method according to one aspect relates to a sensory test and entrusted analysis method performed among a first device, a second device and a third device, the first device being usable by a customer; the second device being usable by a system management company, the third device being usable by an entrusted analysis company including an analytical instrument for analyzing a sample. The sensory test and entrusted analysis method includes: registering, by the second device, information identifying the sample and a sensory evaluation item about the sample; adding, by the second device, identification information to the information identifying the sample; and registering, by the second device, a sensory result generated about the sample.

According to the sensory test and entrusted analysis method of clause 11, the information can be efficiently exchanged among the customer, the system management company and the entrusted analysis company.

(Clause 12) The customer sends, to the system management company, the sample having the identification information added thereto.

According to the sensory test and entrusted analysis method of clause 12, the system management company can receive the sample having the identification information added thereto directly from the customer, and smoothly perform the subsequent processing.

Although the embodiments of the present disclosure have been described, it should be understood that the embodiments disclosed herein are illustrative and non-restrictive in every respect. The scope of the present disclosure is defined by the terms of the claims, and is intended to include any modifications within the scope and meaning equivalent to the terms of the claims.

What is claimed is:

1. A sensory test and entrusted analysis method performed among a first device, a second device and a third device; the first device being usable by a customer, the second device being usable by a system management company, the third device being usable by an entrusted analysis company including an analytical instrument for analyzing a sample, the sensory test and entrusted analysis method comprising:
receiving, by, the second device, information identifying the sample, the information being transmitted from the first device;
registering, by the second device, the information identifying the sample and a sensory evaluation item about the sample;
adding, by the second device, identification information to the information identifying the sample;
transmitting, by the second device, the identification information and an analysis item corresponding to the sensory evaluation item to the third device;
receiving, by the second device, the identification information and an analysis result corresponding to the analysis item of the sample from the third device;
registering, by the second device, a sensory result generated about the sample; and
transmitting, by the second device, the analysis result and the sensory result to the first device.

2. The sensory test and entrusted analysis method according to claim 1, wherein
the sensory evaluation item is selected from a standard sensory evaluation item;
the standard sensory evaluation item is associated with the one or more analysis items,
the second device includes a database that stores the standard sensory evaluation item and the one or more analysis items corresponding to the standard sensory evaluation item in association with each other,
the sensory test and entrusted analysis method further comprising
setting, by the second device, the analysis item corresponding to the sensory evaluation item by using the database.

3. The sensory test and entrusted analysis method according to claim 1, wherein
a fourth device usable by a sensory test company that conducts a sensory test is further provided,
the sensory test and entrusted analysis method further comprising:
transmitting, by the second device, the identification information and a sensory test item corresponding to the sensory evaluation item to the fourth device; and
receiving, by the second device, the sensory result transmitted from the fourth device.

4. The sensory test and entrusted analysis method according to claim 1, wherein
the second device generates the sensory result corresponding to the sensory evaluation item.

5. The sensory test and entrusted analysis method according to claim 1, wherein
the second device includes a machine learning device and a storage device that stores the analysis result in the past as learning data,
the sensory test and entrusted analysis method further comprising:
generating, by the machine learning device, the sensory result by using the learning data; and
transmitting, by the second device, the generated sensory result, to the first device.

6. The sensory test and entrusted analysis method according to claim 1, further comprising:
calculating, by the second device, an amount to be paid, the amount to be paid being an amount to be paid to the entrusted analysis company for the analysis result; and
calculating, by the second device, an amount to be charged based on the amount to be paid, the amount to be charged being an amount to be charged to the customer.

7. The sensor test and entrusted analysis method according to claim further comprising:
receiving, by the second device, an amount to be paid from the third device, the amount to be paid being an amount to be paid to the entrusted analysis company for the analysis result; and
calculating, by the second device, an amount to be charged based on the amount to be paid, the amount to be charged being an amount to be charged to the customer.

8. The sensory test and entrusted analysis method according to claim 3, further comprising:
calculating, by the second device, a first amount to be paid, the first amount to be paid being an amount to be paid to the entrusted analysis company for the analysis result;
calculating, by the second device, a second amount to be paid, the second amount to be paid being an amount to be paid to the sensory test company for the sensory result; and
calculating, by the second device, an amount to be charged based on the first amount to be paid and the second amount to be paid, the amount to be charged being an amount to be charged to the customer.

9. The sensory test and entrusted analysis method according to claim 3, further comprising:
receiving, by the second device, a first amount to be paid from the third device, the first amount to be paid being an amount to be paid to the entrusted analysis company for the analysis result;
receiving, by the second device, a second amount to be paid from the fourth device, the second amount to be paid being an amount to be paid to the sensory test company for the sensory result; and
calculating, by the second device, an amount to be charged based on the first amount to be paid and the second amount to be paid, the amount to be charged being an amount to be charged to the customer.

10. The sensory test and entrusted analysis method according to claim 1, further comprising:
generating, by the second device, a consideration result based on the analysis result and the sensory result, the consideration result indicating a relationship between the analysis result and the sensory result; and
transmitting, by the second device, the consideration result to the first device.

11. A sensory test and entrusted analysis method performed among a first device, a second device and a third device, the first device being usable by a customer, the second device being usable by a system management company, the third device being usable by an entrusted analysis company including an analytical instrument for analyzing a sample, the sensory test and entrusted analysis method comprising:
registering, by the second device, information identifying the sample and a sensory evaluation item about the sample;
adding, by the second device, identification information he information identifying the sample; and
registering, by the second device, a sensory result generated about the sample.

12. The sensory test and entrusted analysis method according to claim 11, wherein
the customer sends, to the system management company, the sample having the identification information added thereto.
